(19) United States
(12) Patent Application Publication
(10) Pub. No.: US 2008/0081072 A1
(43) Pub. Date: Apr. 3, 2008

(54) Resin-complex granulation for water-soluble drugs and associated methods

(76) Inventor: S. Rao Cherukuri, Vienna, VA (US)

Correspondence Address: RICHARD R. MUCCINO, 758 SPRINGFIELD AVENUE, SUMMIT, NJ, 07901, US

(21) Appl. No.: 11/903,074

(22) Filed: Sep. 20, 2007

Related U.S. Application Data
(60) Provisional application No. 60/827,711, filed on Sep. 30, 2006.

(51) Int. Cl. A61K 9/14; A61K 31/135; A61K 31/4045; A61P 9/00; A61K 31/439; A61K 9/10

(52) U.S. Cl. 424/486; 424/497; 424/501; 514/289; 514/415; 514/653

(57) ABSTRACT

The present invention provides methods for preparing pharmaceutical resin-complexed granules which are taste-masked or capable of providing modified release of a water-soluble drug comprising the steps of (a) dissolving a water-soluble drug in water to form a solution; and (b) granulating the drug solution from step (a) in the presence of a resin capable of complexing with the drug to form a drug-resin complex. The drug:resin ratio in step (b) is from about 1:10 to about 10:1, respectively, on a weight/weight basis and the water:resin ratio in step (b) is from about 1:1 to about 5:1, respectively, on a weight/weight basis.

### PRIORITY DATA

This continuation-in-part application claims priority from U.S. provisional patent application Ser. No. 60/827,711, filed on 30 Sep. 2006.

### FIELD OF THE INVENTION

The present invention provides methods for preparing pharmaceutical resin-complexed granules which are taste-masked or capable of providing modified release of a water-soluble drug comprising the steps of (a) dissolving a water-soluble drug in water to form a solution; and (b) granulating the drug solution from step (a) in the presence of a resin capable of complexing with the drug to form a drug-resin complex. The drug:resin ratio in step (b) is from about 1:10 to about 10:1, respectively, on a weight/weight basis and the water:resin ratio in step (b) is from about 1:1 to about 5:1, respectively, on a weight/weight basis.

### BACKGROUND OF THE INVENTION

In the prior art, drug-resin complexes are generally formed by mixing a drug with an aqueous suspension of a resin, after which the complex is then filtered, washed, and dried. These filtering, washing, and drying steps are time consuming and costly.

Accordingly, there is a need for methods for preparing drug-resin complexes without the time consuming and costly steps of filtering, washing, and drying.

### SUMMARY OF THE INVENTION

The present invention provides methods for preparing pharmaceutical resin-complexed granules which are taste-masked or capable of providing modified release of a water-soluble drug comprising the steps of (a) dissolving a water-soluble drug in water to form a solution; and (b) granulating the drug solution from step (a) in the presence of a resin capable of complexing with the drug to form a drug-resin complex. The drug:resin ratio in step (b) is from about 1:10 to about 10:1, respectively, on a weight/weight basis and the water:resin ratio in step (b) is from about 1:1 to about 5:1, respectively, on a weight/weight basis.

### DETAILED DESCRIPTION OF INVENTION

As used herein, the following terms have the given meanings:

The terms “a,” “an,” and, “the” include plural references unless the context clearly dictates otherwise. Thus, for example, reference to “a drug” includes reference to one or more of such drugs, and reference to “an excipient” includes reference' to one or more of such excipients.

The term “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. The term “drug” is expressly encompassed by the present definition as many drugs and prodrugs are known to have specific physiologic activities. These terms of art are well known in the pharmaceutical and medicinal arts.

The term “about” is used to provide flexibility to a numerical range endpoint by providing that a given value may be “a little above” or “a little below” the endpoint.

The term “admixed” means that the drug and/or other ingredients can be dissolved, dispersed, or suspended in the carrier. In some cases, the drug may be uniformly admixed in the carrier.

The term “coating efficiency” refers to the reduction in the amount of coating material needed to coat a given amount of composition to be coated.

The terms “concentrations”, “amounts”, and other “numerical data” may be expressed or presented herein in a range format. Such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

The terms “formulation” and “composition” are used interchangeably and refer to a mixture of two or more compounds, elements, or molecules. In some embodiments the terms “formulation” and “composition” may be used to refer to a mixture of one or more active agents with a carrier or other excipients.

The term “drug”, “pharmaceutically active agent,” “active agent,” and “nutraceutical” may be used interchangeably and refers to a substance that has a measurable physiological effect on a subject when administered thereto.

The term “particle size” refers to the diameter of an individual granular material. Particle sizes are often measured in microns, which are micrometers or one millionth of a meter.

The term “pharmaceutically acceptable carrier” and “carrier” may be used interchangeably, and refer to any inert and pharmaceutically acceptable material that has substantially no biological activity, and makes up a substantial part of the formulation.

The term “pharmaceutically acceptable,” such as pharmaceutically acceptable carriers, excipients, etc., means pharmacologically acceptable and substantially non-toxic to the subject to which the particular compound is administered.

The term “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium, and quaternary ammonium hydroxides, such as for example, tetramethylammonium hydroxide. Chemical modification of a pharmaceutical compound (i.e., drug) into a salt is a technique well known to pharmaceutical chemists to obtain improved physical and chemical stability, hygroscopicity, and solubility of compounds. See, e.g., H. Ansel et. al., Pharmaceutical Dosage Forms and Drug Delivery Systems (6^{th} Ed. 1995) at pp. 196 and 1456-1457.

The terms “plurality of items”, “structural elements”, “compositional elements”, and “materials” may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The term “prodrug” refers to compounds, which undergo biotransformation prior to exhibiting their pharmacological effects. The chemical modification of drugs to overcome pharmaceutical problems has also been termed “drug latentiation.” Drug latentiation is the chemical modification of a biologically active compound to form a new compound, which upon in vivo enzymatic attack will liberate the parent compound. The chemical alterations of the parent compound are such that the change in physicochemical properties will affect the absorption, distribution and enzymatic metabolism. The definition of drug latentiation has also been extended to include nonenzymatic regeneration of the parent compound. Regeneration takes place as a consequence of hydrolytic, dissociative, and other reactions not necessarily enzyme mediated. The terms prodrugs, latentiated drugs, and bio-reversible derivatives are used interchangeably. By inference, latentiation implies a time lag element or time component involved in regenerating the bioactive parent molecule in vivo. The term prodrug is general in that it includes latentiated drug derivatives as well as those substances, which are converted after administration to the actual substance, which combines with receptors. The term prodrug is a generic term for agents, which undergo biotransformation prior to exhibiting their pharmacological actions.

The term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. A composition that is “substantially free of” an ingredient or element may still actually contain such item as long as there is no measurable effect thereof.

The term “therapeutically effective amount” means an amount of a therapeutically effective compound, or a pharmaceutically acceptable salt thereof, which is effective to treat, prevent, alleviate or ameliorate symptoms of a disease.

The term “water-soluble” refers to aqueous solubility and various levels of solubility are described in Remington's Pharmaceutical Sciences and/or Remington: Practice of Pharmacy and/or Martin's Physical Pharmacy. The present invention covers those levels of solubility that are referred to as “very soluble,” “freely-soluble,” “soluble” and “sparingly soluble.”

The present invention provides methods for preparing pharmaceutical resin-complexed granules which are taste-masked or capable of providing modified release of a water-soluble drug comprising the steps of (a) dissolving a water-soluble drug in water to form a solution; and (b) granulating the drug solution from step (a) in the presence of a resin capable of complexing with the drug to form a drug-resin complex. The drug:resin ratio in step (b) is from about 1:10 to about 10:1, respectively, on a weight/weight basis and the water:resin ratio in step (b) is from about 1:1 to about 5:1, respectively, on a weight/weight basis.

The method of the present invention is highly efficient, provides pharmaceutical resin-complexed granules which taste-mask a water-soluble drug and reduces grittiness. Furthermore, this method permits additional coating of the resin-complexed granules with other suitable coating materials such as celluloses or acrylates, and the like.

In one embodiment, the present invention comprises granulation of the water-soluble drug (active agent), wherein the drug is dissolved in an aqueous medium, followed by contacting the drug solution with a complexing resin. Such contacting may be in the form of sprinkling, intimate mixing, grinding, or other techniques in the art. The present invention, in contrast to some prior art methods, does not require filtration of the resin-drug complex after the resin-drug complex has been formed. Therefore this method is much more efficient with increased yields.

The water-soluble drug (pharmaceutically active agent) of the present invention may include any drug belonging to any therapeutic category including but not limited to analgesic, antiallergic, antianxiety, antiasthmatic, antibiotic, anticancer, antidepressant, antidiabetic, antiemetic, anti-inflammatory, antiemetic, anti-Parkinson's, antitussive, antiviral, cardiovascular drugs, and mixtures thereof. The only criterion that may limit the scope of this invention is based on the aqueous solubility of the drug as defined above.

The resins suitable for use in the present invention may be inert organic or inorganic pharmacological resins having a matrix containing covalently bound functional groups that are ionic or capable of being ionized under the appropriate pH conditions. The organic matrix of the resin may be synthetic (e.g., polymers or copolymers of acrylic acid, methacrylic acid, sulfonated styrenes, sulfonated divinylbenzenes) or partially synthetic (e.g., modified celluloses and dextrans). The inorganic matrix of the resin may be silica gel modified by the addition of ionic groups. The covalently bound ionic groups may be strongly acidic (e.g., sulfonic acid), weakly acidic (e.g., carboxylic acid), strongly basic (e.g., quaternary ammonium), weakly basic (e.g., primary amine), or a combination of acidic and basic groups. In general, those types of resins suitable for use in ion exchange chromatography and for such applications as deionization of water are suitable for use in the present invention. Such ion exchangers are described by H. F. Walton in “Principles of Ion Exchange” (pp. 312-343), which disclosure is incorporated by reference herein. The resins useful in the present invention may have exchange capacities below about 6 milliequivalents per gram (meq/g), preferably below about 5.5 meq/g.

The resins in the present invention may be cross-linked with a cross-linking agent selected from difunctional compounds capable of cross-linking polystyrenes. These cross-linking agents are known in the art. Preferably, the cross-linking agent is a divinyl, polyvinyl, or divinylbenzene compound. The resin may be cross-linked to an extent of about 3 to about 20%, preferably about 4 to about 16%, more preferably about 6 to about 10%, and most preferably about 8%, by weight based on the total resin. The resin may be cross-linked with the cross-linking agent by means well known in the art.

The particle size of the resins may range from about 20 μm to about 200 μm. Representative resins useful in this invention include Amberlite™ IRP-69 (obtained from Rohm and Haas) and Dow XYS-40010.00 (obtained from The Dow Chemical Company). Both resins are sulfonated polymers comprised of polystyrene cross-linked with 8% of divinylbenzene, with an ion exchange capacity of about 4.5 to 5.5 meq/g of dry resin (H+-form). Amberlite™ IRP-69 consists of irregularly shaped particles with a size range of 47 μm to 149 μm, produced by milling the parent, large-sized spheres of Amberlite™ IRP-120. The Dow XYS-40010.00 product consists of spherical particles with a size range of 45 μm to 150 μm. Another useful resin, Dow XYS-40013.00, is a polymer composed of polystyrene cross-linked with 8% of divinylbenzene and functionalized with a quaternary ammonium group. Its exchange capacity is normally within the range of approximately 3 to 4 meq/g of dry resin.

In general, the drug:resin ratio will be from about 1:10 to about 10:1, respectively, on a weight/weight basis, and preferably from about 1:2 to about 1:5, respectively, on a weight/weight basis. In some embodiments, the drug:resin ratio may range as follows: about 1:10; about 1:8; about 1:6; about 1:5; about 1:4; about 1:3; about 1:2; about 1:1; about 2:1; about 3:1; about 4:1; about 5:1; about 6:1; about 7:1; about 8:1; about 9:1, respectively, on a weight/weight basis.

The amount of water used in the method for making the pharmaceutical resin-complexed granules of the present invention is generally much lower than that used conventionally. In general, the amount of water used is an amount to dissolve the water-soluble drug and to wet the resin capable of complexing with the drug such that a granulating mass can be obtained. The amount of water necessary to dissolve a particular water-soluble drug varies depending upon the drug and can be readily determined. The amount of water used to dissolve a particular water-soluble drug should be the minimal amount of water necessary but must also be an amount sufficient to wet the resin such that a granulating mass can be obtained. In general, the water:resin ratio to wet a resin will be from about 1:1 to about 5:1, preferably from about 1:1 to 2:1, and more preferably about 1:1, respectively, on a weight/weight basis. In other embodiments, the amount of water is about 2 to 5 times than what is needed to dissolve the drug. Alternatively, the amount of water may be from about 2 to 10 times the amount of resin used on a weight/weight basis. In another embodiment, the amount of water ranges from about 2 to 8 times; or from about 2 to 6 times; or from about 2 to 5 times; or from about 2 to 4 times; or from about 2 to 3 times the amount of resin on a weight/weight basis. In some embodiments, the amount of water is no more than about 10 times the amount of resin on a weight/weight basis.

The lower amount of water used in the present invention offers several advantages: a) the water that is needed to be removed, if any, after formation of the drug-resin complex is minimized which reduces time consuming and costly filtering, washing, and drying methods; b) there is substantially no need to separate (either by filtration or centrifugation or by other separation methods known in the art) the resin-drug complex to isolate the resin-drug complex to be used for further use, instead, the resin-drug complex may be used directly for making granulations offering great savings in time and expenses; and c) lower amounts of water in the drug-resin complex should improve the stability of the complex.

In one embodiment, the invention relates to pharmaceutical compositions comprising drug-resin complexes having only one active ingredient. In another embodiment, the invention relates to pharmaceutical compositions comprising the drug-resin complexes in combination with pharmaceutically acceptable non-toxic carriers or excipients. In one embodiment, the drug is selected from the group consisting of an antihistamine, a sympathomimetic drug (nasal decongestant, bronchodilator), analgesic, anti-inflammatory, cough suppressant and/or expectorant. In one embodiment, the cough suppressant is dextromethorphan or dimehydrinate or codeine. Compounds which are antihistamines, sympathomimetic drugs (nasal decongestant, bronchodilator), analgesic, anti-inflammatory, cough suppressants and/or expectorants are well known to those of skill in the art.

In some embodiments, the drug-resin complexes are non-coated. In other embodiments, the drug-resin complexes are coated. In one embodiment, from about 20% to about 80% of the drug-resin complex in the composition is coated, most preferably about 40% to about 60% of the drug-resin complex. The coating may be a water-permeable, diffusion barrier coating material. The presence of a coating allows one to selectively modify the dissolution profile as desired of a pharmaceutical composition comprising the drug-resin complexes of the present invention.

The coating materials can in general be any of a large number of conventional natural or synthetic film-forming materials used singly, or in mixtures thereof, and in admixture with plasticizers, pigments, etc. with diffusion barrier properties and with no inherent pharmacological or toxic properties. In general, the major components of the coating may be water-insoluble or permeable to water and a drug. However, it might be desirable to incorporate a water-soluble substance, such as methyl cellulose, to alter the permeability of the coating, or to incorporate an acid-insoluble, base-soluble substance to act as an enteric coating. The coating materials may be applied as a suspension in an aqueous fluid or as a solution in organic solvents. Suitable examples of such coating materials are described by R. C. Rowe in Materials used in Pharmaceutical Formulation. (A. T. Florence, editor), Blackwell Scientific Publications, Oxford, 1-36 (1984), which disclosure is incorporated by reference herein. Preferably the water-permeable diffusion barrier is selected from the group consisting of ethyl cellulose, methyl cellulose, and mixtures thereof. The coating material may be for example, Surelease®, manufactured by Colorcon, which is water-based ethyl cellulose latex, plasticized with dibutyl sebacate or with vegetable oils. Other non-limiting coating materials included within the scope of the present invention are Aquacoat®, manufactured by FMC Corporation of Philadelphia, Pa., which is an ethyl cellulose pseudolatex; solvent based ethyl cellulose; shellac; zein; rosin esters; cellulose acetate; Eudragits®, manufactured by Rohm and Haas of Philadelphia, Pa., which are acrylic resins, silicone elastomers, poly(vinyl chloride) methyl cellulose, and hydroxypropylmethyl cellulose.

Conventional coating solvents and coating procedures (such as fluid bed coating and spray coating) can be employed to coat the particles. Fluid bed coating is disclosed, for example, in U.S. Pat. Nos. 3,089,824, 3,117,027, and 3,253,944. The coating is normally applied to the drug-resin complex, but alternatively can be applied to the resin before mixing the resin with the drug. Non-limiting examples of coating solvents include ethanol, mixtures of methylene chloride and acetone, coating emulsions, methyl acetone, tetrahydrofuran, carbon tetrachloride, methyl ethyl ketone, ethylene dichloride, trichloroethylene, hexane, methyl alcohol, isopropyl alcohol, methyl isobutyl ketone, toluene, 2-nitropropane, xylene, isobutyl alcohol, and n-butyl acetate.

The coated drug-resin complexes may be coated in the range from about 40% to about 70% w/w drug-resin complex, preferably, from about 45% to about 55% w/w drug-resin complex, more preferably, about 50% w/w drug-resin complex. Variations in the amount of coating and/or the use of coated/uncoated mixtures can be employed to selectively modify the dissolution profile as desired.

The average particle size of the non-hydrated coated and uncoated drug-resin complexes may range from about 60 μm to about 200 μm and about 60 μm to about 250 μm, respectively. In one embodiment, average particle sizes of the coated drug-resin complexes may range from about 70 μm to about 190 μm, or may be from about 70 μm to about 180 μm. In another embodiment, average particle sizes of the uncoated drug-resin complexes may range from about 55 μm to about 160 μm, or from about 60 μm to about 150 μm. It is desirable that about 85%, preferably about 95%, and most preferably about 98% of the resin particles have sizes within the ranges set forth above. Adjustments within these ranges can be made to accommodate desired aesthetic qualities of the final formulation product.

Additional advantages achieved by the pharmaceutical resin-complexed granules of the present invention relate to taste. Dextromethorphan is a drug, which is bitter and unpleasant to take orally. Compositions, such as a liquid suspension, comprising the pharmaceutical resin-complexed granules of the present invention surprisingly are pleasant tasting with good mouth-feel, even in the absence of sugars.

The drug-resin complex of the invention may be stored for future use or formulated with conventional pharmaceutically acceptable carriers to prepare liquid compositions. The drug-resin complexes according to this invention may, for example, take the form of liquid preparations such as suspensions or solid preparations such as capsules, tablets, caplets, liquigells, and powders.

Aqueous suspensions may be obtained by dispersing the drug-resin complexes in a suitable aqueous vehicle, optionally with the addition of suitable viscosity enhancing agent(s) (e.g., cellulose derivatives, xanthan gum, etc.). Non-aqueous suspensions may be obtained by dispersing the drug-resin complexes in a suitable non-aqueous based vehicle, optionally with the addition of suitable viscosity enhancing agent(s) (e.g., hydrogenated edible fats, aluminum stearate, etc.). Suitable non-aqueous vehicles include, for example, almond oil, arachis oil, soybean oil or fractionated vegetable oils such as fractionated coconut oil.

The compositions may be formulated using conventional carriers or excipients and well-established techniques. Illustrative non-limiting examples include conventional carriers or excipients include diluents, binders and adhesives (i.e., cellulose derivatives and acrylic derivatives), lubricants (i.e., magnesium or calcium stearate, or vegetable oils, polyethylene glycols, talc, sodium lauryl sulphate, polyoxy ethylene monostearate), solubilizers, humectants, disintegrants, colorants, flavorings, preservatives, sweeteners and miscellaneous materials such as buffers and adsorbents in order to prepare a particular medicated composition.

Suitable thickeners include: tragacanth; xanthan gum; bentonite; acacia and lower alkyl ethers of cellulose (including the hydroxy and carboxy derivatives of the cellulose ethers). Preferably, tragacanth is used and incorporated in an amount of from about 0.1% w/v to about 1.0% w/v of the composition, and more preferably about 0.5% w/v of the composition. Xanthan gum is used in the amount of from about 0.025% w/v to about 0.5% w/v, preferably about 0.25% w/v.

Suitable humectants useful in the formulations of the present invention include glycerin, polyethylene glycol, propylene glycol and mixtures thereof. Preferably, polyethylene glycol is used and incorporated in an amount of from about 5% w/v to about 20% w/v of the composition and preferably in an amount of from about 5% w/v to about 15% w/v of the composition and most preferably in an amount of about 8% w/v of the composition.

The oral liquid compositions of the present invention will also comprise at least one and preferably two surfactants in amounts of up to about 5.0% w/v and preferably from about 0.02% w/v to about 3.0% w/v of the total formulation. The surfactants useful in the preparation of the compositions of the present invention are generally organic materials, which aid in the stabilization and dispersion of the ingredients in aqueous systems for a suitable homogenous composition. Preferably, the surfactants of choice are non-ionic surfactants such as poly(oxyethylenesorbitan monooleate (Tweens® 80) and sorbitan monooleate (Spans® 80). These surfactants are commercially produced in a wide variety of structures and molecular weights. While many surfactants may be used, preferably a compound from the group comprising polysorbate copolymers (sorbitan-mono-9-octadecenoate-poly(oxy-1,2-ethanediyl)) is employed. This compound is also added and functions to keep many flavors and sweeteners homogeneously dissolved and dispersed in solution. It is also believed, without being bound to theory, that the polymers may provide a taste masking function as well by binding with the active ingredient. Suitable polysorbates include polysorbate 20, polysorbate 40, polysorbate 80 and mixtures thereof. In one specific embodiment, polysorbate 80 is employed. The surfactant component will comprise from about 0.01% w/v to about 2.0% w/v of the total composition and preferably will comprise about 0.1% w/v of the total weight of the composition.

A second emulsifier/surfactant useful in combination with polysorbates in the practice of the present invention may be employed and is preferably a poloxamer such as Poloxamer 407. Polyxamer 407 has an HLB (hydrophilic/lipophilic balance) of about 22 and is sold under the trade name Pluoronic®-127 (BASF-Wyandotte; Parsippany, N.J.). The two surfactants can be employed in substantially equivalent amounts. For example, the Poloxamer 407 and polysorbate 80 may each be employed together at levels of approximately from about 0.02% w/v to about 4.0% w/v of the total weight of the composition.

Preservatives useful in the present invention include, but are not limited to, sodium benzoate, potassium sorbate, ethylenediaminetetraacetic acid (EDTA), and parabens (e.g., methyl, ethyl, propyl or butyl-hydroxybenzoates, etc.) or sorbic acid. The preservatives listed above are exemplary, but each preservative must be evaluated on an empirical basis, in each formulation, to assure the compatibility and efficacy of the preservative. Methods for evaluating the efficacy of preservatives in pharmaceutical formulations are known. Preferred preservatives are the paraben preservatives such as methyl, ethyl, propyl, and butyl paraben. Methyl and propyl paraben are most preferable. Preferably, both methyl and propyl paraben are present in the formulation in a ratio of methyl paraben to propyl paraben of from about 2.5:1 to about 7.5:1, preferably 3:1

The pharmaceutical resin-complexed granules of the present invention can be prepared according to the examples set out below. The examples are presented for purposes of demonstrating, but not limiting, the preparation of the dosage forms of this invention.

### EXAMPLES

### Example 1

Phenylephrine pharmaceutical resin-complexed granules were prepared according to the method of the present invention. The ingredients are set out below.

| SL. NO. | INGREDIENT | QUANTITY |
|---|---|---|
| 1. | Phenylephrine HCl | 100 g |
| 2. | Amberlite ™ IRP 88N | 200 g |
| 3. | Purified water | 200 g |
| 4. | Ethyl cellulose 4 cps | 15.8 g |
| 5. | Isopropyl alcohol | 61 g |

Phenylephrine (Neo-Synephrine) is an α-adrenergic receptor agonist used primarily as a decongestant. Phenylephrine HCl was dissolved in purified water in a suitable container with stirring. Amberlite™ IRP88N was passed through #30 mesh and loaded into a rapid mixing granulating bowl. The drug solution was added slowly to the Amberlite™ IRP88N in a high shear mixer granulator. The mixture was thoroughly mixed for about 15 minutes or until it became a uniform wet mass. The vessel was rinsed with purified water and the rinse was added to the bowel. The wet mass was dried in a fluid bed drier for 2 hours at an inlet temperature of 50° C. The dried drug-resin complex was removed from the fluid bed drier into pre-weighed polybag lined bins. Isopropyl alcohol was added to ethyl cellulose 4 cps without forming lumps by stirring until a clear solution was formed. The drug-resin complex was granulated with a polymer solution by adding slowly to the complex under mixing in a rapid mixing granulator. The mixture was thoroughly mixed for approximately ten minutes or until it became a uniform wet mass. The wet mass was loaded into a fluid bed drier bowl and dried for 1.5 hours at an inlet temperature of 50° C. These granules were then compressed into tablets.

### Example 2

Following the method set out in Example 1, drug-resin complexes of sumatriptan hydrochloride granules were prepared making appropriate changes to the formulation of Example 1. Sumatriptan is a triptan drug having a sulfonamide group, which is useful for the treatment of migraine headaches. Thus, sumatriptan 25 mg, 50 mg, and 100 mg were prepared that were taste-masked with an Amberlite™ IRP88N resin.

### Example 3

Following the method set out in Example 1, drug-resin complexes of guaifenesin granules were prepared by making appropriate changes to the formulation of Example 1. Guaifenesin is an expectorant drug usually taken orally to assist the expectoration (“bringing up”) of phlegm from the airways in acute respiratory tract infections. Thus, guaifenesin 300 mg and guaifenesin 600 mg were prepared that were taste-masked with an Amberlite™ IRP88N resin or with Amberlite™ IRP69 resin.

### Example 4

Following the method set out in Example 1, drug-resin complexes of dextromethorphan hydrobromide granules were prepared. Dextromethorphan is an antitussive (cough-suppressant) drug found in many over-the-counter cold and cough medicines. The composition of this Example is set out below.

| SL. NO. | INGREDIENTS | QUANTITY |
|---|---|---|
| 1. | Dextromethorphan HBr | 33.3 g |
| 2. | Amberlite ™ IRP 69 | 71.7 g |
| 3. | Purified water | 500 g |

Similarly, dextromethorphan 15 mg and 60 mg were prepared. The higher dose was then further coated with ethyl cellulose 4CPS, according to the procedure set out in Example 1.

### Example 5

Following the method set out in Example 1, drug-resin complexes of clopidogrel bisulphate granules were prepared by making appropriate changes to the formulation of Example 1. Clopidogrel is a potent oral anti-platelet agent used in the treatment of coronary artery disease, peripheral vascular disease, and cerebrovascular disease. Thus, clopidogrel 50 mg, 75 mg and 100 mg were prepared that were taste-masked with an Amberlite™ IRP88N resin.

### Example 6

### Part A: Drug-Resin Granulation

Phenylephrine HCl (100 grams) was dissolved in 200 grams of purified water in a suitable container with stirring. Pass 200 grams of resin polacrillin potassium, methacrylic acid and divinylbenzene polymer (Amberlite™ IRP88N) through #30 mesh and load into RMG Bowl. Add slowly drug solution to the resin Amberlite™ IRP88N in high shear mixer granulator. Mix by switching ON impeller set at 75 rpm for approximately fifteen minutes or until it becomes an uniform wet mass. Rinse the vessel with purified water and add the rinsing to the bowel. Immediately dry the wet mass in a fluid bed drier.

Load the wet mass into a fluid bed drier bowl. Dry for 2 hours at an inlet temperature of 50° C. and observe the LOD at 105° C. for 2 min. (Limit not more than 7.5%). Pass the dried mass through #40 mesh and if required dry further till target moisture content is obtained.

### Part B: Polymer Granulation

A quantity of 61 grams of isopropyl alcohol was added to a suitable container and stirred to form a vortex. To the vortex add 15.8 grams of ethyl cellulose 4 cps without forming lumps. Stir until a clear solution is formed. Load drug resin complex into RMG bowl. Granulate drug resin complex with polymer solution by adding slowly to the complex powder under mixing. Mix by switching ON impeller set at 75 rpm for approximately ten minutes or till it becomes an uniform wet mass. Use additional isopropyl alcohol to obtain a proper wet mass. Immediately dry the wet mass in a fluid bed drier.

Load the wet mass from above into a fluid bed drier bowl. Dry for 1.5 hours at an inlet temperature of 50° C. and observe the LOD at 105° C. for 2 min. (Limit: Not more than 7.5%).

Remove the dried granules from the fluid bed drier into pre-weighed polybag lined bins. Pass the dried mass through # 40 mesh and if required dry further till target moisture content is obtained

Analytical data:

| Test | | | | |
|---|---|---|---|---|
| Number | Specifications/test | Limits | Batch 1 | Batch 2 |
| 1 | Assay | NLT 90.0% and NMT | 95.6% | 94.0% |
| | | 110.0% | | |
| 2 | Content uniformity | Average: NLT 90.0% | 95.2% | 93.5% |
| | | and NMT 110.0% of | | |
| | | label Claim | | |
| | | NLT 90.0% of | MIN: | MIN: |
| | | Average Assay | 94.6% (Limit: NLT | 92.1% (Limit: NLT |
| | | | 85.7%) | 84.2%) |
| | | NMT 110.0% of | MAX: | MAX: |
| | | Average Assay | 96.1% (Limit: NMT | 95.0% (Limit: NMT |
| | | | 104.7%) | 102.9%) |
| | | RSD ≦ 6.0% | RSD: 0.5% | RSD: 1.3% |
| 3 | Residual solvent | Less Than 3000 ppm | 19.97 ppm | 33.88 ppm |
| 4 | Dissolution profile(Ph 1.2 | NLT 75% | 96.0% | 94.0% |
| | buffer, 900 ml, paddle, 50 RPM, | | | |
| | 37 C, 30 minutes | | | |
| 5 | Moisture content | NMT 9.0% | 7.3% | 8.9% |
| 6 | Particle size analysis- | | | |
| | specifications | | | |
| 6.1 | Percentage retained on#80 | NMT 10.0% | 6.2% | 4.2% |
| | mesh | | | |
| 6.2 | Percentage retained on | NMT 10.0% | 3.7% | 2.5% |
| | #100 mesh | | | |
| 6.3 | Percentage retained on # | NLT 30.0% & NMT | 62.1% | 45.4% |
| | 200 mesh | 65.0% | | |

Stability data: Microencapsulated Phenylephrine HCl granules 32%

| | | | | | | Related substances | |
|---|---|---|---|---|---|---|---|
| | | | | | | (By HPLC) % w/w | |
| | | | Loss on | | Assay | Individual | Total |
| Conditions | Time Point | Description | drying | Dissolution | % of label claim | Maxima | Impurities |
| Initial | | WWGP | 7.88 | 90.5%-97.10% | 97.55 | — | — |
| 25° C. ± 2° C. and | 1 month | WWGP | 9.24 | 95.1%-99.24% | 97.50 | — | — |
| 60% RH ± 5% | 2 months | WWGP | 10.46 | 93.96%-98.44% | 97.17 | — | — |
| | 3 months | WWGP | 10.85 | 100.18%-101.94% | 96.69 | — | — |
| | 7 months | WWGP | 11.03 | — | — | 0.065 | 0.184 |
| | 11 months | WWGP | 11.32 | — | — | 0.077 | 0.210 |
| 40° C. ± 2° C. and | 1 month | WWGP | 9.63 | 94.63%-96.21% | 97.15 | — | — |
| 75% RH ± 5% | 2 months | WWGP | 10.87 | 92.52%-94.52% | 96.67 | — | — |
| | 3 months | WWGP | 11.52 | 93.70%-100.34% | 96.20 | — | — |
| | 7 months | WWGP | 11.52 | — | — | 0.09 | 0.203 |
| | 11 months | WWGP | 12.57 | — | — | 0.133 | 0.256 |

WWGP: White to off white, odorless granular powder

### Example 7

### Part A: Drug-Resin Granulation

Phenylephrine HCl (50 grams) was dissolved in 200 grams of purified water, in a suitable container, with stirring. Pass 200 grams of resin polacrillin potassium, methacrylic acid and divinylbenzene polymer (Amberlite™ IRP88N) through #30 mesh, pass 50 grams of Microcrystalline cellulose through a #30 mesh and load both into a RMG Bowl. Switch on the RMG bowl and mix both the ingredients for 5 minutes at 75 RPM. Add slowly drug solution to the resin Amberlite™ IRP88N in high shear mixer granulator. Mix with impeller set at 75 rpm for approximately fifteen minutes or until it becomes a uniform wet mass. Rinse the vessel with purified water and add the rinsing to the bowel. Immediately dry the wet mass in a fluid bed drier.

Load the wet mass into fluid bed drier bowl. Dry for 2 hours at an inlet temperature of 50° C. and observe the LOD at 105° C. for 2 min. (Limit: not more than 7.5%). Pass the dried mass through # 40 mesh and if required dry further till target moisture content is obtained.

### Part B: Polymer Granulation

A quantity of 61 grams of isopropyl alcohol was added to a suitable and stirred to form vortex. To the vortex add 15.8 grams of ethyl cellulose 4 cps without forming lumps. Stir until clear solution is formed. Load drug resin complex into RMG Bowl. Granulate drug resin complex with polymer solution by adding slowly to the complex powder under mixing. Mix by switching ON impeller set at 75 rpm for approximately ten minutes or until it becomes an uniform wet mass. Use additional isopropyl alcohol to obtain a proper wet mass. Immediately dry the wet mass in a fluid bed drier.

Load the wet mass from above into fluid bed drier bowl. Dry for 1.5 hours at an inlet temperature of 50° C. and observe the LOD at 105° C. for 2 min. (Limit: Not more than 7.5%). If required dry further until target moisture content is obtained

Remove the dried granules from fluid bed drier into pre-weighed polybag lined bins. Pass the dried mass through # 40 mesh. Optionally 1.5 g of silicon dioxide can be added during sifting to avoid rupture of the film, facilitate screening, remove static charges in the encapsulation (which causes agglomeration), and enhance the stability of the encapsulation.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

We claim:

1. A method for preparing pharmaceutical resin-complexed granules which are taste-masked or capable of providing modified release of a water-soluble drug comprising the steps of:
(a) dissolving a water-soluble drug in water to form a solution; and
(b) granulating the drug solution from step (a) in the presence of a resin capable of complexing with the drug to form a drug-resin complex;
wherein the drug:resin ratio in step (b) is from about 1:10 to about 10:1, respectively, on a weight/weight basis and the water:resin ratio in step (b) is from about 1:1 to about 5:1, respectively, on a weight/weight basis.

2. The method of claim 1, wherein the drug in step (a) is selected from the group consisting of analgesic, antiallergic, antianxiety, antiasthmatic, antibiotic, anticancer, antidepressant, antidiabetic, antiemetic, anti-inflammatory, antiemetic, anti-Parkinson's, antitussive, antiviral, cardiovascular drugs, and mixtures thereof.

3. The method of claim 2, wherein the drug is selected from the group consisting of: phenylephrine, dextromethorphan, sumatriptan, and their pharmaceutically acceptable salts and mixtures thereof.

4. The method of claim 1, wherein the resin in step (b) is selected from the group consisting of polymers or copolymers of acrylic acid, sulfonated styrenes, sulfonated divinylbenzenes, modified celluloses, dextrans, silica gels modified by the addition of ionic groups, and cross-linked resins of the foregoing wherein the cross-linking agent is a difunctional compound capable of cross-linking polystyrenes.

5. The method of claim 4, wherein the resin is selected from the group consisting of sulfonated polymers comprised of polystyrenes cross-linked with 8% of divinylbenzene, with an ion exchange capacity of about 4.5 to about 5.5 meq/g of dry resin (H+-form), polymers comprised of polystyrene cross-linked with 8% of divinylbenzene and functionalized with a quaternary ammonium group with an exchange capacity of about 3 to 4 meq/g of dry resin, and mixtures thereof.

6. The method of claim 1, wherein the particle size of the resin ranges from about 20 μm to about 200 μm.

7. The method of claim 1, wherein the drug:resin ratio in step (b) is from about 1:2 to about 1:5, respectively, on a weight/weight basis.

8. The method of claim 1, wherein the water:resin ratio in step (b) is from about 1:1 to 2:1, respectively, on a weight/weight basis.

9. The method of claim 1, wherein the water:resin ratio in step (b) is about 1:1, respectively, on a weight/weight basis.

10. The method of claim 1, wherein the drug-resin complex is further granulated or coated with a cellulose polymer, an acrylate polymer, a wax, an emulsifier, and mixtures thereof, in a non-aqueous medium.

11. The method of claim 10, wherein the drug-resin complex is not separated prior to granulation.

12. The method of claim 1, wherein the drug-resin complex granules are further processed into a pharmaceutical unit dosage form selected from the group consisting of capsules, tablets, caplets, films, orally disintegrating dosage forms, chewable tablets, and modified release dosage forms.
